(19) United States
(12) Patent Application Publication
(10) Pub. No.: US 2014/0030734 A1
(43) Pub. Date: Jan. 30, 2014

(54) Biomarkers for Diseases of the Central Nervous System

(71) Applicants: Antonio Bertolotto, Torino (IT); Italiana Sclerosi Multipla - FISM Onlus, Genova (IT)

(72) Inventors: Antonio Bertolotto, Torino (IT); Simona Perga, Torino (IT); Alessandra Giuliano Albo, Torino (IT); Davide Corpillo, Torino (IT)

(21) Appl. No.: 13/896,382

(22) Filed: May 17, 2013

(30) Foreign Application Priority Data
May 18, 2012 (IT) MI2012A000865

(51) Int. Cl. G01N 33/68

(52) U.S. Cl. CPC G01N 33/6893
USPC 435/7.4; 436/501; 435/7.92; 435/7.94; 435/320.1

(57) ABSTRACT

The present invention describes a method of diagnosis and/or progression of a disease based on the detection and quantification of the expression levels of biomarkers preferably selected from the group comprising gelsolin isoforms (SEQ. ID 1 and 2) and Vitamin D binding protein isoforms (SEQ. ID 3 and 4). Said method is applied to central nervous system diseases, preferably to Multiple Sclerosis.

The present invention discloses a diagnosis and/or progression method of a disease based on the detection and quantification of the expression levels of biomarkers preferably selected from the group comprising gelsolin isoforms (SEQ. ID 1 and 2) and Vitamin D binding protein isoforms (SEQ. ID 3 and 4). Said method is applied to central nervous system diseases, preferably to Multiple Sclerosis.

### STATE OF THE ART

The inflammatory and degenerative diseases affecting the central nervous system comprise a wide range of diseases for which very often, to date, no efficient treatment therapy is available. For many of these, a diagnosis is also particularly difficult, but when the disease is full blown, when the therapeutic symptomatic approaches are most of times less efficient than the same therapies introduced in the onset stage. A further major limitation is the difficulty in monitoring the disease progression, which progression often has a wide heterogeneity in the various afflicted subjects; this difficulty translates into the impossibility of monitoring in an objective manner the effectiveness of the therapeutic approach in the particular individual.

Particular important among those diseases affecting the central nervous system is Multiple Sclerosis (MS). MS is a demyelinating chronic inflammatory disease, with autoimmune pathogenesis, affecting the central nervous system (brain and spinal cord).

The natural history of the disease is heterogeneous. Onset symptoms may occur, individually or in combination, in an acute form that completely or partially relapse, or in a slowly progressive form. It may mainly involve the sensory-motor, visual systems, and the cerebellar/vestibular system (Ghezzi et al., 1980). Usually, at a variable time interval, re-exacerbation phases may occur, combining the most various combinations of symptoms: the most recurrent remain those of the onset, with the particularity that, upon time, outcomes tend to become fixed, showing other disorders besides to the initial ones. In the most advanced phases, the disease progression becomes stable, compromising a number of important functional systems. The wide variability in the symptoms characterizing it is due to a degeneration process of myelin, the protective sheath covering and isolating the nervous fibers and allowing the quick and integral transmission of signals thereby. In the course of the disease, the destruction of myelin sheaths causes the block or slowing of the pulses going from the central nervous system to different areas in the body, and vice versa. The areas in which myelin was damaged are referred also to as plaques; hence the definition of plaque sclerosis.

The disease often affects subjects aged 20-40years, even if the onset of the disease in children—teenagers (before 15 years) and after 50 years is getting more and more frequent. The prevalence for women is clear, with a F/M ratio of 2:1; prevalence data in Italy show a ratio of 50/90 for every 100,000 population.

MS onset is determined by a recurrent, immune-based inflammatory process, damaging myelin, and secondly to the damage to the latter, also to the axons contained therein, which irreversibly deteriorate, forming a base for the progressive permanent disability. Such demyelination phenomena are diffused in the white matter of the Central Nervous System (CNS), encephalon, and marrow, and give rise to a variety of signs and symptoms reflecting the different localization of brain, marrow, or optical nerve focal lesions.

The symptoms of the disease depend on the localization of the demyelination areas, which represent the most important histo-pathologic lesion in MS; however, the appearance of symptoms can be caused by both the edema and the action of toxic inflammatory mediators, and the axonal loss. The progressive damage to the axons, in cases with a chronic course, leads to an extensive degeneration and cerebral atrophy, which seems to be strongly related to permanent neurological deficits, in a higher extent to demyelination (Poser et al., 1985).

Etiology of the disease is still unknown, while its pathogenesis seems to be related to a combination of various factors. MS can be considered as a disease that is paradigmatic of a set of pathological conditions currently known as multifactorial diseases, where genetic and environmental factors interact in a complex manner.

In 1996 the definitions for the different clinical courses of MS were standardized:

relapsing-remitting MS (MSRR),

secondary progressive MS (MSSP),

primary progressive MS (MSPP).

MSRR is characteristics of patients having an initial exacerbation, followed by a complete or partial recovery. Although approximately 85% of patients with MS follows this disease course from its onset, 50% within ten years, and 90% after 25 years develops a gradual progression of disability that can be accompanied by relapses or not; in such a case, the disease is referred to as secondary progressive MS (MSSP). 10-15% of patients shows a gradual progression of the disability from the onset, which is not accompanied by exacerbations: this is the primary progressive (MSPP) form. Finally, a new term, progressive relapsing MS (MSPR), was introduced to describe patients with a disease course that is progressive from the onset, subsequently accompanied by one or more relapses with a worsening in the progression in the intervals between relapses (Lublin and Reindgold, 1996).

Predicting the clinical course of the disease is difficult, considered the large interindividual variability of the clinical course (from benign, even asymptomatic cases, to clinical forms with more severe signs).

To date, there is still no specific therapy for MS. Pharmacological treatments target acute episodes, the prevention of relapses, and a general improvement of symptoms.

The diagnosis of MS is not easy, above all because, upon its onset, the disease does not show characterizing symptoms; instead, in most of cases they appear suddenly and are generally non-specific, and could also be the result of other, less severe diseases. To reach a MS diagnosis, to date, no test is available, which is able to confirm in a definite and indisputable manner the diagnosis of MS; however, the latter is given by a clinician based on three elements: i) the symptoms complained of by the patient; ii) neurological examination; iii) positive outcome of some instrumental examinations, among which mainly nuclear magnetic resonance (NMR). NMR is a sensitive and specific examination that is able to determine the cerebral and medullary lesions responsible for the various neurological signs and symptoms of MS. However, the appearance of the lesions is a late event occurring after the pathogenetic molecular events.

The need to have an early diagnosis method is strongly felt, since it would allow a timely ad correct therapeutical approach. A number of studies showed that exactly an early pharmacological treatment allows reducing by 50% the risk of developing this disease, besides decreasing the aggressiveness thereof and slowing its progression (Freedman, 2011). Anticipating, predicting and blocking are the three keywords to avoid the progression of the disease.

Currently, no biochemical—molecular tool is available, allowing the physicians early identifying MS patients and recognizing their clinical type. In the practice, only the presence in the cerebrospinal fluid of oligoclonal Immunoglobulins IgG is usually used as a biochemical tool for supporting the clinical diagnosis of Multiple Sclerosis.

The biomedical research on MS has, among its priorities, the identification of specific biomarkers associated to the disease and the different manifestations thereof. In literature, the involvement in MS of gelsolin is disclosed, which is a multifunctional protein capable of bonding actin; in particular, decreases in the levels of gelsolin in the cerebrospinal fluid (CSF) of MS patients were observed (Kulakowska et al., 2008, 2010). In pediatric MS patients, instead, an increase in the levels of gelsolin and also of Vitamin D binding protein (DBP) was shown (Rithidech et al., 2009). Further studies pointed out that DBP levels in the CSF are lower during remission stages, to then increase in the secondary progressive stage (Disanto et al., 2011). PCT/SE2006/000866 proposes to assess the levels of phosphorilated DBP as a diagnostic tool to distinguish MS patients affected by the RR form, from those with a SP form.

In spite of the large number of studies about it, to date, biomarkers that are not only qualitative, but also quantitative, associated to MS and that can be used clinically are not available. In particular, to date, biomarkers capable of distinguishing, upon the diagnosis, those patients who will develop a full-blown form of MS from those who will develop other neurological diseases are not available, and markers for the progression of the disease that may help the physician in prescribing a more suitable pharmacological treatment are not available either. A not timely or not suitable pharmacological intervention is one of the reasons for which the available treatments show a effectiveness that is limited and variable from patient to patient. Moreover, it shall be noticed that, in the absence of efficient therapies, a prompt diagnosis would allow starting the treatments in advance, then to prolong life expectancy and quality of the patient (Lacomblez et al., 1996). Furthermore, the absence of biomarkers does not allow an efficient classification on a molecular basis of the different phenotypes of MS, which is a complex multifactorial disease that probably encompasses within its clinical definition various neurological disorders (Shaw and Williams, 2000; Bowser et. al., 2006). On the other hand, the absence of specific biomarkers, reflects poor knowledge of the molecular mechanisms involved in the onset and development of MS, with the result that, in testing new compounds for therapeutic action, it is not possible to obtain a quantitative indication of the effectiveness thereof. To this effect, the discovery of new biomarkers may concur to the understanding of the disease mechanisms, and therefore to the development of possible new therapeutic targets.

Between the bodily fluids that can be analyzed in order to identify MS biomarkers, CSF represent a unique source of information relative to the disease and its pathogenetic mechanisms, since it is secreted by CNS structures and contains peptides, proteolytic fragments, and antibodies that may reflect the presence and progression of the disease. Therefore, the discovery of new biomolecules in the CSF of affected patients may improve the diagnostic discrimination within the heterogeneous extent of the disease. In particular, the identification of biomarkers capable of distinguishing clinically relevant MS subgroups would represent a powerful, promising tool to determine prognostic factors, to monitor the clinical course in different MS phenotypes, as well as to identify therapeutic targets in function of a customized cure.

To the light of what has been pointed out above, the urgent need to find MS-specific biomarkers useful for the diagnosis, formulation of the prognosis, and the monitoring of the disease progression in its different definitions will be apparent. Therefore, the object of the present invention is to provide a method for diagnosis, progression, and prognosis of MS based on the individuation and identification of specific biomarkers. Said biomarkers turn out to be useful not only for MS; but also for other inflammatory and degenerative diseases affecting the central nervous system.

### DESCRIPTION OF THE INVENTION

By a proteomic analysis carried out on biologic fluid samples obtained by subjects affected by inflammatory and degenerative diseases of the central nervous system, markers were shown, the trend of which is surprisingly quantitatively related to the disease.

### DESCRIPTION OF THE FIGURES

FIG. 1: bidimensional gel, two spots are shown, relative to gelsolin isoforms precursors (ID 97, 98) and two spots relative to two DBP isoforms (ID 288, 289).

FIG. 2: dendrogram resulting from the cluster analysis performed on the average values of the percent volumes (Vol %) of the most representative spots of the analyzed gel population (250 spots), two groups are shown, A and B;

FIG. 3: dendrogram resulting from the cluster analysis performed by selecting the set of biomarkers included in the panel 1 described, in Table 3, two groups are shown, A1 and B1;

FIG. 4: trend of the average Vol %±SD of the spots ID 97, 98, and 289 in the two clusters A1 and B1;

FIG. 5: levels of the average Vol % of the spots ID 288and 289 in the individual patients included in the study;

FIG. 6: dendrogram obtained from the cluster analysis based on the Vol % of the spots ID 288 and 289, two groups are shown, G and Z.

FIG. 7: combination of the values of the Vol % of the spots ID 288 and 289 in the groups G and Z.

FIG. 8: dendrogram resulting from the cluster analysis based on the Vol % of the spots ID 97, 98, 288 and 289.

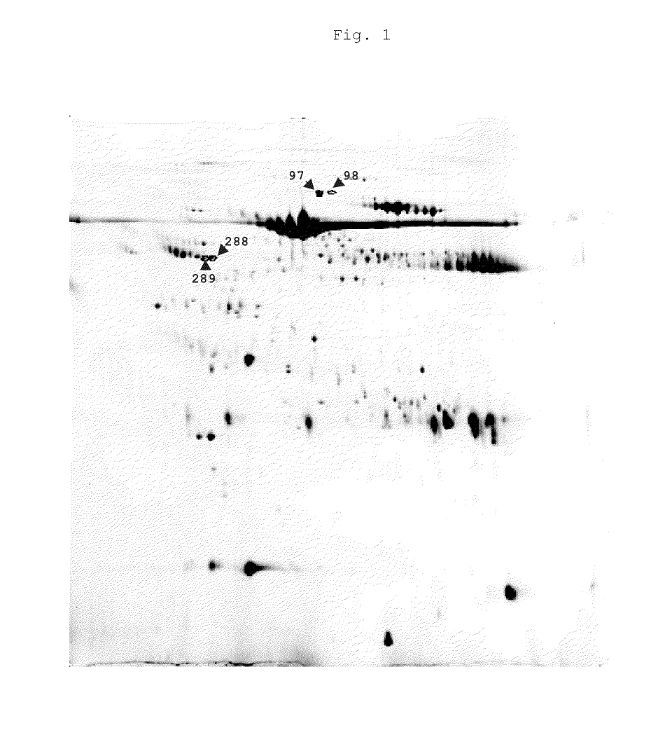

### DETAILED DESCRIPTION OF THE INVENTION

Unless explicitly indicated otherwise, the following terms used in the present application are meant in the meaning indicated herein below:
“percentage of identity” and “% of identity” between two amino acid (peptides) or nucleic acid sequences (nucleotides): percentage of amino acid or nucleotide acid residues that are identical in corresponding positions in the two sequences optimally aligned. In order to determine the “percentage of identity” of the two amino acid or nucleic acid sequences, the sequences are aligned together; to get an optimal comparison, interruptions can be introduced in the sequences (i.e., deletions or insertions—which can optionally be located also at the end of the sequences) (gap). The amino acid and nucleotidic acid residues in corresponding positions are then compared. When a position in the first sequence is occupied by the same amino acid or nucleotide residue that occupy the corresponding position in the second sequence, the molecules are identical in that position. The percentage of identity between two sequences is as a function of the number of identical positions shared by the sequences [i.e., % of identity=(number of the identical positions/total number of the positions)×100]. According to an advantageous embodiment, the sequences have the same length. Advantageously, the compared sequences do not have interruptions (or insertions).
The percentage of identity can be obtained by using mathematical algorithms. A non-limiting example of a mathematical algorithm used for the comparison of two sequences is the Karlin and Altschul algorithm [Proc. Natl. Acad. Sci. USA 87 (1990) 2264-2268] modified by Karlin and Altschul [Proc. Natl. Acad. Sci. USA 90 (1993) 5873-5877]. Such algorithm is incorporated in the BLASTn and BLASTp programs by Altschul [Altschul, et al., J. Mol. Biol. 215 (1990) 403-410].
In order to obtain alignments even in the presence of one or more interruptions (or insertions), it is possible to use methods allocating a relatively high penalty to each interruption (or insertion), and a lower penalty for each additional amino acid or nucleotide residue in the interruption (such additional amino acid or nucleotide residue is defined as an extension of the interruption). It shall be apparent that high penalties will determine optimized alignments with a lower number of interruptions. When using the BLAST programs, the BLOSUM62 matrix is typically employed.
An advantageous, non-limiting example of a program to implement an optimal alignment is the GCG Wisconsin Bestfit package (University of Wisconsin, USA; Devereux et al., 1984, Nucleic Acids Research 12:387). Also in this case, the default parameters are used that, for an amino acid sequence, provide for a penalty of −12 for an interruption, and a penalty of −4 for each extension.
By “percentage of homology” and “% of homology” between two amino acid or nucleic acid sequences is meant the percentage of homologous amino acid or nucleotide residues in corresponding positions in the two optimally aligned sequences. The percentage of homology between two sequences is determined in a manner that is substantially identical to what has been described above for determining the percentage of identity, except in that, in the computation, also the homologous positions are considered, not only the identical positions. As regards a nucleotide sequence, two homologous positions have two different nucleotides, but which within their own codon lead to the codification of a same amino acid. As regards an amino acid sequence, two homologous positions have two homologous amino acids, i.e., amino acids that are provided with similar chemical-physical properties, for example, amino acids belonging to the same groups, such as: aromatic (Phe, Trp, Tyr), acid (Glu, Asp), polar (Gln, Asn), basic (Lys, Arg, His), aliphatic (Ala, Leu, Ile, Val) groups, with a hydroxyl group (Ser, Thr), with a short side chain (Gly, Ala, Ser, Thr, Met). It is expected that substitutions between such homologous amino acids do not alter the protein phenotype (conservative substitutions of amino acids). Specific examples of conservative substitutions are known in this technical field, and are described in literature [e.g., Bowie et al., Science, 247:1306-1310 (1990)].
“Corresponding position”—a position in a sequence of a polypeptide or nucleic acids corresponding to (facing), following an alignment, a determined position of a reference sequence.
By “level” is meant the level of expression of a protein in the sample, as measured with one of the techniques as described herein and/or known to those skilled in the art.

It is the object of the present invention the identification of polypeptides that are present in biologic fluids of patients affected from autoimmune neurological disease, degenerative diseases of the central nervous system, inflammatory diseases of the central nervous system, the expression levels of which can be related to well-defined clinical pictures and pathologic phenotypes. Preferably, said diseases will be selected from Multiple Sclerosis, the neuromyelitis optica (Devich's disease), encephalites associated with auto-antibodies, among which encephalites associated with anti-NMDA antibodies and anti-GAD antibodies (Stiff Person syndrome), paraneoplastic syndromes of the central nervous system with auto-antibodies (for example anti-Tr antibodies; Alzheimer's disease, Parkinson's disease; viral or bacterial central nervous system infections (encephalites and meningites), vasculitides, demyelinating diseases. Said polypeptides were identified by known proteomics techniques, to be then identified by means of mass spectrometry techniques. The identification of said peptides is set forth in the following Table 1.

TABLE 1
Mass spectrometry identification of some of the selected spots.

| | | | | AA | | Theoretical | |
|---|---|---|---|---|---|---|---|
| Spot | LC-MS/MS | Accession | Peptide | coverage | Sequence | MW | Mascot |
| ID | identification | No. | No. | (%) | coverage | (Das)/pI | score |
| 70 | Complement | gi 291922 | 10 | 18 | 51-696 | 85.4/6.5 | 514 |
| | factor B | | | | | | |
| 72 | Complement | gi 291922 | 12 | 18 | 183-707 | 85.4/6.5 | 222 |
| | factor B | | | | | | |
| 97 | Gelsolin isoform | gi 4504165 | 21 | 37 | 33-748 | 85.6/5.9 | 415 |
| | precursor | | | | | | |
| 98 | Gelsolin isoform | gi 4504165 | 6 | 10 | 148-738 | 85.6/5.9 | 117 |
| | precursor | | | | | | |
| 99 | Afamin | gi 4501987 | 8 | 14 | 89-453 | 69/5.6 | 52 |
| | precursor | | | | | | |
| 101 | Serum | gi | 6 | 10 | 25-508 | 74.6/6.6 | 159 |
| | transferrin | 110590597 | | | | | |
| 102 | Serum | gi | 18 | 29 | 25-674 | 74.6/6.6 | 346 |
| | transferrin | 110590597 | | | | | |
| 117 | Glycoprotein | gi 69990 | 3 | 6 | 86-415 | 51.9/5.6 | 77 |
| | alpha 1-β | | | | | | |
| 227 | Albumin | gi 28592 | 3 | 5 | 427-581 | 69.3/6 | 56 |
| | fragment | | | | | | |
| 288 | Vitamin D- | gi 181482 | 13 | 30 | 52-440 | 53.0/5.4 | 291 |
| | binding protein | | | | | | |
| 289 | Vitamin D- | gi 181482 | 20 | 56 | 52-471 | 52.9/5.4 | 480 |
| | binding protein | | | | | | |
| 469 | Apolipoprotein E | gi 178853 | 14 | 50 | 20-317 | 36.1/5.8 | 348 |

In particular, it is pointed out that the two polypeptides ID 288 and 289 represent modified DBP isoforms. Considering the migration of the two spots, since an increase in glycosylation is known to result in a migration to a more acidic position, the spot ID 289, being to the left side compared to the spot ID 288, would be a glycosylated isoform of DBP.

The biomarkers identified in the present invention can be detected and quantified by techniques that are known to those skilled in the art for the analysis of proteins, selected preferably from the group comprising Western-Blot, 2DE gel electrophoresis, ELISA (Enzyme-Linked Immunosorbent assay), RIA (Radioimmunoassay), Competitive EIA (Competitive Enzyme Immunoassay), DAS-ELISA (Double Antibody Sandwich-ELISA), other immunocytochemical or immunohistochemical techniques. Said techniques will be combined, as necessary and as well known to those skilled in the art, with HPLC or mass spectrometry. In a preferred embodiment, said assessments are carried out by 2DE gel electrophoresis.

In particular, it has been pointed out that the levels of ID 97, ID 98, and ID 289 are increased in those patients affected by a pathological form that will develop into a more severe course. On the other hand, ID 288 is increased in patients affected by a pathological form with a slower, less aggressive progression. Surprisingly, the present analysis has also shown that the values of the ID 288 and ID 289 levels are inversely related. In particular, the values of the ID 289 and ID 288 levels were combined together by means of the linear functions f1 and f2:

f1=33.813*(ID 288 level)+48.942*(ID 289 level)−7.155;

f2=103.508*(ID 288 level)+42.871*(ID 289 level)−15.248.

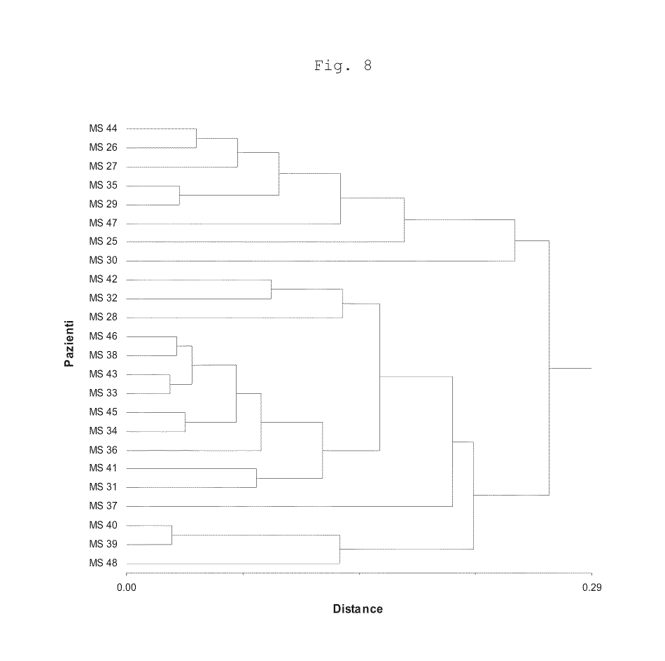

by illustrating in a graph the values that the two functions take based on the levels measured in each of the analyzed samples, a good separation of the two populations, clusters G and Z, (FIG. 7) has been observed, and the straight line capable of separating them was described by the function y=1.5629x−1.7155. Values exceeding the straight line defined by the function y=1.5629x−1.7155 are associated with a less severe progression of the disease (cluster G); those values that are below the same straight line are associated to a more severe progression (cluster Z). Therefore, the function y=1.5629x−1.7155 turns out to be able to stratify the population of samples based on the severity of the disease progression.

It is claimed herein a method of diagnosis and progression of Multiple Sclerosis comprising:
i. measuring the ID 289 (SEQ. ID 4) and ID 288 (SEQ. ID 3) level in a sample C;
ii. obtaining the coordinates f1 and f2, where f1=33.813*ID 288 level+48.942*ID 289 level−7.155 and f2=103.508*ID 288 level+42.871*ID 289 level−15.248;
iii. positioning in the Cartesian plane the point C(f1, f2) and assessing the position thereof relative to the straight line y=1.5629x−1;
iv. where C(f1, f2)>y=1.5629x−1, the sample C will be assigned to the MSSP or MSPP group; where C(f1, f2)<y=1.5629x−1, the sample C will be assigned to the MSRR group.
   Where said assessments are carried out by 2DE gel electrophoresis, said levels are related to the Vol % of the spots measured for ID 288 and ID289, respectively.

Furthermore, it has been noticed that the Vol % values of the spots ID97 and ID98 are capable of distinguishing between MSSP or MSPP and MSRR. It is a further object of the present invention a method comprising the assessment of the levels of the gelsolin isoforms ID97 (SEQ. ID 1) and/or ID98 (SEQ. ID 2) in the sample and the comparison of the same levels assessed in a subject affected by MSRR. Said levels of the gelsolin isoforms ID97 (SEQ. ID 1) and ID98 (SEQ. ID 2) are assessed by a method capable of distinguishing between the two isoforms, or by a method capable of assessing the two isoforms in a combined manner or, alternatively, of assessing also a single isoform. By G, the value of said levels of the gelsolin isoforms is herein defined, whether it is obtained from the sum of the isoforms ID97and ID98 assessed independently, or from the same isoforms assessed in a combined manner or, alternatively, only of the isoform ID 97 or only of the isoform ID98. Where the value measured in the sample was increased in a statistically significant manner with respect to the value measured in the MSRR subject, said sample will be assigned to the MSSP-MSPP group. Where the value measured in the sample does not depart in a statistically significant manner from the value measured in the MSRR subject, said sample will be assigned to the MSRR group.

A method of assessment of the progression of Multiple Sclerosis is herein claimed, comprising:
i. measuring the ID 97 (SEQ. ID 1) and/or ID 98 (SEQ. ID 2) level in a sample C and in a control MSRR;
ii. obtaining the value G=ID 97 level+ID 98 level, or G=ID 97 level or G=ID 98 level in the sample G_{C} and in the control G_{CtrlMSRR};
iii. where G_{C}>G_{CtrlMSRR} in a statistically significant manner, assigning the sample C to the MSSP or MSPP group; where G_{C}=G_{Ctrl}, the sample C will be assigned to the group MSRR.

In a further embodiment, the method of diagnosis and progression claimed herein comprises:
i. measuring the ID 97 (SEQ. ID 1) and/or ID 98 (SEQ. ID 2) level in a sample C and in a control MSRR, and measuring the ID 289 (SEQ. ID 4) and ID 288(SEQ. ID 3) level in the sample C;
ii. obtaining the coordinates f1 and f2, where f1=33.813*ID 288 level+48.942*ID 289 level−7.155 and f2=103.508*ID 288 level+42.871*ID 289 level−15.248;
iii. positioning in the Cartesian plane the point C(f1, f2) and assessing the position thereof relative to the straight line y=1.5629x−1;
iv. obtaining the value G=ID 97 level+ID 98 level, or G=ID 97 level G=ID 98 level in the sample (G_{C}) and in the control (G_{CtrlMSRR});
v. where C(f1, f2)>y=1.5629x−1 and G_{C}>G_{Ctrl} in a statistically significant manner, assigning the sample C to the MSSP or MSPP group; where C(f1, f2)<y=1.5629x−1 and G_{C}=G_{CtrlMSRR}, assigning the sample C to the MSRR group.
   It is a further object of the present invention a method of diagnosis and progression of the disease, comprising obtaining the coordinates f1 and f2 as defined above and/or of the value G as defined above and the combination of the assessment of C(f1, f2) and/or G with the assessment of further biomarkers selected, by way of illustrative, non-limiting example, in the group comprising: complement factor C3, complement factor C4, beta-2-microglobulin, Clusterin, Prostaglandin H2 D isomerase, Haptoglobin, Immunoglobulins, Apolipoprotein E, Beta fibrinogen, alpha 2 macroglobulin, Fetuin A, serin protease inhibitor, Tansthyretin, Albumin, Transferrin, Apolipoprotein D, Retinol binding protein, Apolipoprotein A4, SPARC-like protein, Autotaxin t, Pigment epithelium derived factor, Angiotensinogen, Chromogranin A, Tuberous sclerosis complex 2, Cystatin C, Ceruloplasmin, Superoxide dismutase 1, Actin, Beta V Spectrin, Cartilage acidic protein 1, Fibulin 1, Calsyntenin 3, Contactin 1, Xantin dehydrogenase/oxidase, RNA binding motif protein 7, Ribonuclease 1, neuroendocrine protein 7 B2, alpha 1 Antitrypsin, alpha 1 Antichymotrypsin, Kallikrein 6, EGF-containing fibulin-like extracellular matrix protein 1, Plasminogen, Antithrombin III, Dickkofp relata protein 3, neuronal pentraxin receptor, Tetranectin, so that the combination of C(f1, f2) and/or G with one or more of said biomarkers results in obtaining enhanced predictive statistical power. It shall be apparent that such combinations fall within the object and scope of the instant patent, as well as any other combinations of C(f1, f2) and/or G with other biomarkers and/or physiological and/or diagnostic markers.

It is a further object of the present invention a kit for implementing the methods described above, the kit comprising two antibodies capable of specifically recognizing the two isoforms of Vitamin D Binding protein SEQ. ID 3 and SEQ. ID 4, and/or one or two antibodies capable of recognizing in a combined or selective manner the gelsolin isoforms SEQ. ID 1 and SEQ. ID 2 and, optionally, one or more antibodies capable of recognizing one or more of the selected biomarkers, by way of illustrative, non-limiting example, in the group comprising: complement factor C3, complement factor C4, beta-2-microglobulin, Clusterin, Prostaglandin H2 D isomerase, Haptoglobin, Immunoglobulins, Apolipoprotein E, Beta fibrinogen, alpha 2 microglobulin, Fetuin A, serin protease inhibitor, Tansthyretin, Albumin, Transferrin, Apolipoprotein D, Retinol binding protein, Apolipoprotein A4, SPARC-like protein, Autotaxin t, Pigment epithelium derived factor, Angiotensinogen, Chromogranin A, Tuberous sclerosis complex 2, Cystatin C, Ceruloplasmin, Superoxide dismutase 1, Actin, Beta V Spectrin, Cartilage acidic protein 1, Fibulin 1, Calsyntenin 3, Contactin 1, Xantin dehydrogenase/oxidase, RNA binding motif protein 7, Ribonuclease 1, neuroendocrine protein 7 B2, alpha 1 Antitrypsin, alpha 1 Antichymotrypsin, Kallikrein 6, EGF-containing fibulin-like extracellular matrix protein 1, Plasminogen, Antithrombin III, Dickkofp relata protein 3, neuronal pentraxin receptor, Tetranectin. Said kit may comprise also reagents for the detection of said antibodies bound to said proteins.

The method claimed herein, in its various embodiments, offers an objectivity that would not be otherwise obtained with the clinical methods currently in use for the diagnosis, progression, and prognosis of the disease, the method being claimed herein as exclusively related to a molecular aspect of the disease and the measurement of quantitative parameters.

It is a further object of the present invention the induction of the isoform of the Vitamin D binding protein SEQ. ID4, such induction being obtained by acting on the metabolic processes resulting in the post-translational modifications observed in the spot ID289 with respect to the spot ID 288. In particular, the induction of the enzyme sialidase is herein claimed, which is obtained by an increased synthesis, a decreased catabolism and/or, a provision of co-factors. In a preferred embodiment, said induction of the enzyme sialidase is obtained by a recombinant vector selected from the vectors that are integrated or not integrated in the genome, characterized in that said recombinant vector comprises a gene coding for sialidase or an homologous thereof, or contains an element capable of acting at the level of the endogenous promoter of said sialidase by activating it. Furthermore, the use of inducers of galactosidases is claimed, in particular of Beta galactosidase and N acetyl galactosaminidase. Said inducers are preferably selected from the group comprising isopropyl-b-D-thiogalactopyranoside (IPGT) and lactose. In a further embodiment, an inhibition of the enzyme glucosyltransferase is claimed, obtained preferably with Interferon.

A composition is also claimed, comprising one or more of the compounds listed above and pharmacologically acceptable excipients for use in the treatment of MS.

### EXAMPLES

### Example 1

### Sample Collection

CSF samples of a homogeneous population of 24 women affected by Multiple Sclerosis were collected. The clinical data of the patients enrolled for this study are described in detail in the following Table 2.

TABLE 2
Clinical data of the patients MS enrolled in the instant study.

| | | CSF | Serum | | | CSF | | |
|---|---|---|---|---|---|---|---|---|
| RRMS | | protein | protein | CSF IgG | Serum IgG | Albumin | IgG | |
| patients | OCB | (mg/dl) | (mg/dl) | (mg/dl) | (mg/dl) | (mg/dl) | Index | Cells/μl |
| MS 25 | tras | 20 | 683 | 0.585 | 443.7 | 8.7 | 0.44 | 3 |
| MS 26 | pos | 34 | 1190 | 3.52 | 345.6 | 15.7 | 0.65 | 5 |
| MS 27 | pos++ | 36 | 1467 | 11.5 | 378.5 | 14.4 | 2.1 | 28 |
| MS 28 | pos++ | 72 | 909 | 7.32 | 382.7 | x | 0.5 | 20 |
| MS 29 | pos++ | 30 | 1562 | 5.08 | 494.5 | 16.9 | 1 | 16 |
| MS 30 | pos++ | 22 | 1475 | 3.72 | 436.4 | 9.3 | 1.18 | 20 |
| MS 31 | pos++ | 35 | 1170 | 18.7 | 548.4 | 13.4 | 6.5 | 45 |
| MS 32 | pos++ | 36 | 634 | 3.38 | 360.1 | 13.7 | 1.4 | 8 |
| MS 33 | pos++ | 35 | 790 | 6.21 | 443.8 | 24.1 | 1.4 | 38 |
| MS 34 | pos++ | 33 | 920 | 7.22 | 448.6 | 23.6 | 1.5 | 16 |
| MS 35 | pos | 25 | 892 | 2.75 | 437.8 | 21 | 0.6 | 3 |
| MS 36 | pos++ | 38 | 1002 | 7.67 | 325.8 | 17.4 | 1.4 | 5 |
| MS 37 | pos++ | 29 | 1072 | 3.39 | 338.7 | 19.2 | 0.6 | 3 |
| MS 38 | pos++ | 21 | 988 | 3.32 | 362.2 | 12.6 | 0.1 | 8 |
| MS 39 | pos++ | 59 | 903 | 4.96 | 349 | 39.1 | 0.5 | 3 |
| MS 40 | tras | 49 | 1133 | 3.03 | 522.7 | 22.2 | 0.63 | 3 |
| MS 41 | neg | 36 | 1241 | 3.51 | 297.5 | 23.5 | 0.4 | 3 |
| MS 42 | neg | 32 | 1023 | 1.62 | 397.8 | 23.5 | 0.3 | 2 |
| MS 43 | pos++ | 46 | 1463 | 6.74 | 383.8 | 27.6 | 0.6 | 8 |
| MS 44 | pos++ | 40 | 1310 | 2.27 | 372.3 | 33.5 | 0.2 | 2 |
| MS 45 | neg | 43 | 1052 | 3.66 | 402 | 33.1 | 0.42 | 2 |
| MS 46 | pos++ | 35 | 955 | 4.7 | 346.1 | 20.7 | 0.82 | 8 |
| MS 47 | pos++ | 29 | 1103 | 4.39 | 432.7 | 17.3 | 1 | 6 |
| MS 48 | pos | 31 | 644 | 1.61 | 334.5 | 23.6 | 0.4 | 2 |

Legenda:
CSF: Cerebro Spinal Fluid
OCB: Oligo Clonal Band

Upon the collection, said patients were affected by a RR form of Multiple Sclerosis according to the clinical criteria established by McDonald's (2001).

The average age of the subjects included in the study upon the liquor collection was about 36 years: the youngest subject was 17 years old, while the oldest one was 61 years old.

The liquor samples were collected in the remitting phase, and all the subjects, except for the patients MS 27, MS 29, MS 35, MS 25, and MS 26, were not undergoing a pharmacological treatment.

The lumber puncture for CSF withdrawal was performed between the fourth and fifth intervertebral disk space according to a procedure that allowed withdrawing a liquor volume of 20 mL. The CSF samples obtained from each subject were collected in propylene tubes in the presence of protease inhibitors, centrifuged at 1,000×g at 4° C. for 10 minutes, and finally stored at −80° C., until the time of analysis.

In order to allow the protein extraction, the biological samples were subjected to a process of concentration and elimination of salts by means of special devices, having a 5 kDa filter (Ultrafree Millipore, Bedford, USA) according to the procedures indicated in the use manual. Albumin was not removed from the samples, not only because albumin is a transport protein that can bind markers of interest, but also because modified forms of albumin differentially present in the disease of interest may play a role for diagnostic purposes. Therefore, the removal of albumin may lead to the loss of useful biomarkers.

### Example 2

### Follow Up

The patients were monitored in the two years following the collection, except for the patients MS 40 and MS 43.

### Example 3

### 2DE and Statistical Analysis

On the samples prepared as in the Example 1, a 2DE experiment was carried out at least in duplicate, according to the procedure described by Robotti et al. 2010, with some modifications. For each sample, 100 μg total protein was heated for 5 min at 95° C. in the presence of 10 μl sodium dodecylsulfate (SDS) at 5% (w/v) and dithiothreitol (DTT) at 2.5% (w/v), and then diluted to 330 μl with a buffer containing urea (7 M), thiourea (2 M), 3[(3-Colamidopropyl)dimethylammonium]-propansulfonic acid (CHAPS) at 4% (w/v), IPG 3-10NL buffer at 0.5% (v/v) (GE Healthcare, Uppsala, Sweden), and traces of bromophenol blue. The sample was then loaded on 18 cm non-linear strips for isoelectrofocusing (IEF) from IPG 3-10 (GE Healthcare) with rehydratation in gel (2 h at 0 V, and 12 h at 30 V). A IEF was carried out at 20° C. with IPGphor apparatus (GE Healthcare), according to the following protocol: 500 V at 500 V/h, 1.000 V at 1.000 V/h with linear gradient; 8,000 V at 13,500 V/h with linear gradient; 8,000 V at 72,000 V/h. Before the denaturating electrophoresis in gel of polyacrylamide-SDS (SDS-PAGE), the IPG strips were equilibrates twice for 15 min in Tris-HCl buffer (50 mM) at pH 8.8, urea (6 M), 30% glycerol (v/v), SDS al 2% (w/v) and traces bromophenol blue, containing 1% DTT (w/v) for the first passage and 2.5% iodoacetamide (w/v) for the second passage. This was then followed by SDS-PAGE in a 12.5% gel (1.5 mm thickness), according to the Laemmli protocol [1970], but without stacking gel, by using a Hoefer SE600 apparatus (GE Healthcare). The second dimension was stroke at 60 ma/gel at 16° C., and it was stopped when the front part of the bromophenol blue reached the lower end of the gel. For the calibration of the molecular weights (MW) and of the isoelectric points (pI) proteins having a MW ranging between 15 and 100 kDa and with a pI ranging between 4.5and 8.5 were used as standard. The gels were stained with Sypro Ruby (Molecular Probes Inc, Eugene, Oreg.). After staining, digital images of the gel were taken by using a ProXPRESS 2D CCD camera (Perkin Elmer). The images were analyzed by the ImageMaster 2D Platinum 5.0 software (GE Healthcare). A “detection” of the spots of each image was performed, then only one reference gel was selected, i.e., the gel containing the highest number of correctly focused spots. Each gel was then virtually overlapped (“matching”) on the reference gel. The matching was automatically performed by the software; subsequently, it was manually corrected. For each spot, Vol % was calculated as the integral of the volume of each spot stained with Sypro (area of the spot multiplied by its intensity) normalized on the sum of the volumes of all the spots present in the gel. The data relative to the most representative % volumes of the spots, i.e., those present at least in 80% of the analyzed population (250 spots) were exported in an Excel worksheet, and for each of them the average Vol % value obtained from at least two technical replicates was calculated. These data have been subjected to a cluster analysis by using the StatistiXL software version 1.x (CustomCD, http://www.statisticxl.com). The used approach provided for the use of a hierarchical cluster analysis, in which a hierarchy of partitions was built, which were characterized by a decreasing/increasing number of groups, visible by a tree representation (dendrogram), in which the grouping/division steps of the groups are represented. The Euclidean distance was considered as the similarity measure.

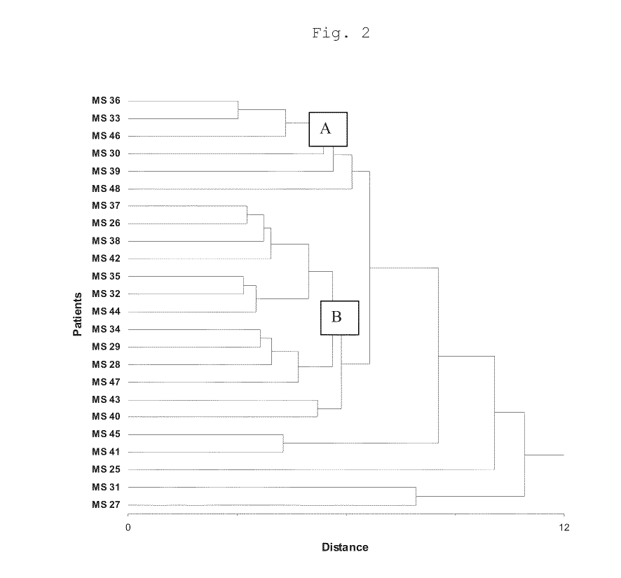

From this cluster analysis, two main subgroups of patients were identified (Cluster A and Cluster B), and 5 outlier subjects (FIG. 2). In order to establish which spots were most responsible for this grouping into clusters, a statistical analysis (Mann-Whitney test, with a significance level (P) equal to or less than 0.05, by using the GraphPad software version Instat 3.00) and of fold-change criterion (>1.5) were applied. With this double approach, two panels of spots were identified: a first panel consisting in 3 spots (panel 1) and a second panel consisting in 3 spots (panel 2) (Table 3).

TABLE 3

| | | | | | Fold- |
|---|---|---|---|---|---|
| Spot | | Cluster A | Cluster B | P Value | change |
| ID | Panel | (% Vol) | (% Vol) | (<0.05) | (>1.5) |
| 97 | 1 and 2 | 0.125 ± 0.035 | 0.210 ± 0.057 | <0.01 | ↑ 1.7 |
| 98 | 1 | 0.033 ± 0.003 | 0.046 ± 0.011 | <0.05 | ≅ |
| 288 | 2 | 0.224 ± 0.150 | 0.139 ± 0.099 | NS | ↓ 1.6 |
| 289 | 1 and 2 | 0.121 ± 0.025 | 0.214 ± 0.082 | <0.05 | ↑ 1.7 |

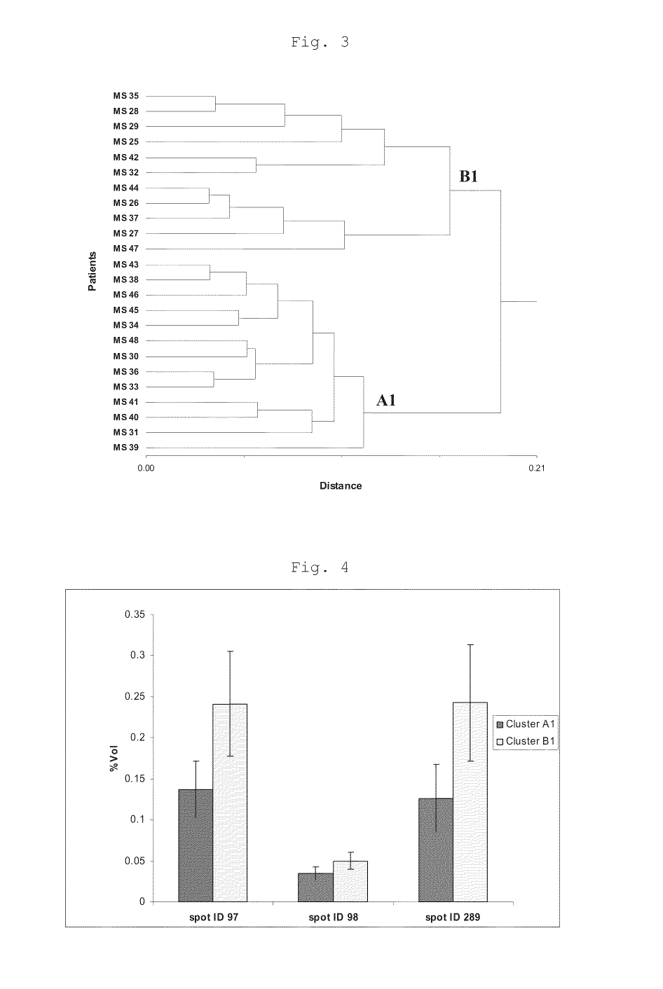

Subsequently, a further clusterization was performed by selecting the values of the Vol % of the spots of the panel 1. Following this analysis, a dendrogram is obtained, where the patients were grouped into two different clusters, renamed cluster A1 and cluster B1 (FIG. 3). These two new clusters are very similar to those obtained from the previous study (FIG. 2), except for the patients MS 34, MS 38, MS 40, and MS 43 who were moved from cluster B to cluster A (renamed Cluster B1 and Cluster A1, respectively). As regards outliers, MS 41, MS 31, and MS 4 5 are grouped in the cluster A1, while outliers MS 25 and MS 27 are found in cluster B1. The average values of the Vol % that the spot ID 98, 97, and 289 take in these 2 new clusters are set forth in FIG. 4.

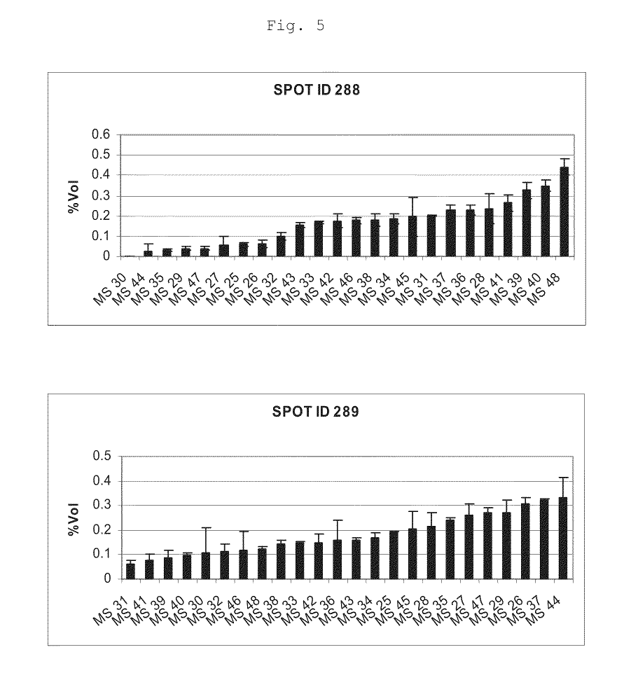

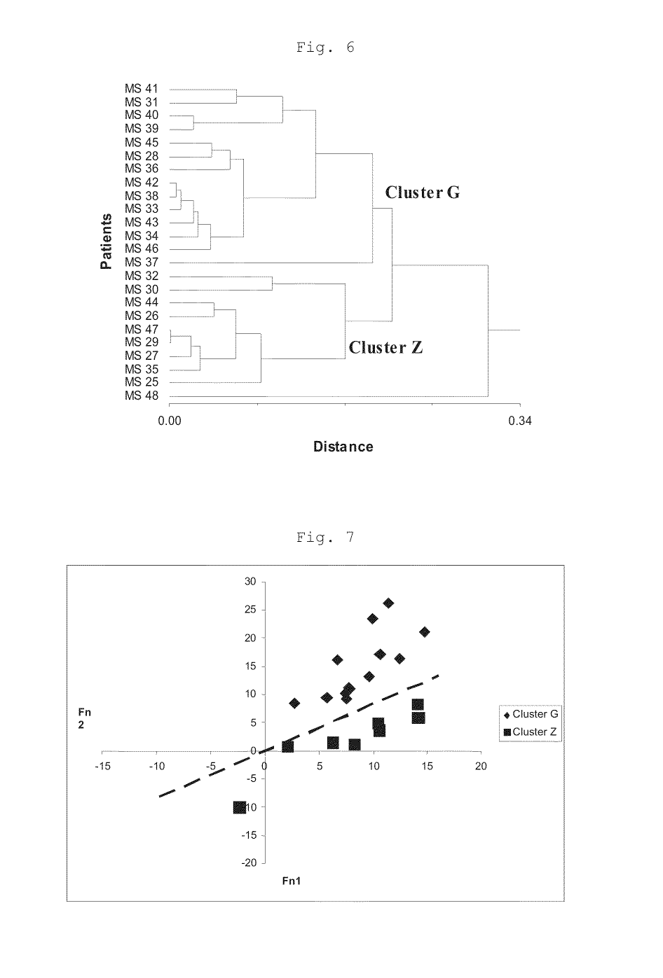

By performing the cluster analysis only on the spots ID 288 and 289, the detailed results of which for each patient are set forth in FIG. 5, a dendrogram was obtained, in which two main clusters are represented, referred to as Cluster G and Cluster Z and an outlier, patient MS 48 (FIG. 6). Furthermore, again with reference to the outlined spots ID 288 and 289, a post-test discriminant analysis was performed, to test the ability thereof to classify each subject in the pertinent population based on the volumes % measured (used software: StatistiXL). The overall ability of a correct prediction reaches 100% (FIG. 7).

### Example 4

### Biomarkers-Disease Relationship

All the patients included in the cluster B1 in the Example 3, except for the female patients MS 29, MS42 and MS 25, have a delta EDSS>0, and after a two-year follow-up are subjected to therapy. EDSS variation was obtained by comparing the EDSS score upon lumber puncture and after two-year follow-up. All the patients included in the cluster A1 after two years are not so far undergoing any treatments, and the EDSS value remained unaltered post-collection.

Therefore, the patients included in the group B1 show a more aggressive disease course; on the contrary, patients included in the cluster A1 show a more benign disease course.

### Example 5

### Mass Spectrometry Identification of the Identified Biomarkers

The spot identified in Example 3 were manually cut from the corresponding gels and discolored overnight with a solution containing 40% ethanol in ammonium bicarbonate (25 mM); then they were washed twice with ammonium bicarbonate (25 mM), thrice with acetonitrile, then dried. Each gel fragment was rehydrated in ammonium bicarbonate (25 mM) containing 0.6 μg modified porcine trypsin, and a protease digestion was carried out overnight at 37° C. The resulting peptides were extracted by sonication in ammonium bicarbonate (25 mM), loaded on a ZORBAX 300 SB C18 RP column (75 μm×150 mm, 3.5 μm particles, Agilent, Milan, Italy), and eluted with a 5%-80% acetonitrile gradient (containing 0.1% formic acid) at a 0.3 μl/min flow rate, by using a HP 1100 nanoLC system coupled to a XCT-Plus nanospray-ion trap mass spectrometer (Agilent) (LC-ESI MS/MS). The parameters used for the mass spectrometer were as follows: scan width=100-2,200 m/z; scan rate=8,100 m/z per s; gas flow rate=5 l/min; temperature=300° C.; capillary=1.8 kV; skimmer=40 V; ion charge control target (ICC)=125,000; maximum build-up time=300 ms. The positively charged peptides were automatically isolated and fragmented, and spectra were deconvoluted by using the DataAnalysis software (Bruker Daltonics, Bremen, Germany). The LC-ESI MS/MS mass data were used for searching the non-redundant NCBI protein sequence database through the Mascot research algorithm (http://www.matrixscience.com—mass tolerance of the monoisotopic peak: 1.8 Da for the parental ion, or 0.8 Da for the fragments; maximum number of uncut sites per peptide of 3). Carbamidomethylation of cysteines and oxidation of methionines were considered as permitted changes. Results with a Mowse score exceeding 47 were considered as significant (p<0.05) (Mila et. al., 2009).

The protein identity of the thus-obtained biomarkers is set forth in the following Table 4 and indicated by the corresponding sequence identifiers (SEQ ID NO).

TABLE 4

| | | | | Number | | Apparent | |
|---|---|---|---|---|---|---|---|
| SEQ. | Spot | | Accession | of | Coverage | MW(KDa)/ | |
| ID | ID | LC-ESI MS/MS | number | Peptides | sequence | pI | Mowse |
| 1 | 97 | Gelsolin | gi4504165 | 21 | 33-748 | 85.6/5.9 | 415 |
| | | Isoform | | | | | |
| | | Precursor | | | | | |
| 2 | 98 | Gelsolin | gi4504165 | 6 | 148-738 | 85.6/5.9 | 117 |
| | | Isoform | | | | | |
| | | Precursor | | | | | |
| 3 | 288 | Vitamin D- | gi181482 | 13 | 52-440 | 53.0/5.4 | 291 |
| | | binding | | | | | |
| | | protein | | | | | |
| 4 | 289 | Vitamin D- | gi181482 | 20 | 52-471 | 52.9/5.4 | 480 |
| | | binding | | | | | |
| | | protein | | | | | |

Mass spectrometry analysis allowed identifying the spots ID 97 and 98 as gelsolin (SEQ. ID 1 and 2), and both spots ID 288 and 289 turn out to be attributable to DBP (SEQ. ID 3 and 4).

The following Tables set forth the protein sequence for the proteins identified in the spots ID 97, 98, 288, and 289.

| Spot 97 SEQ. ID 1 | |
|---|---|
| 1 | MAPHRPAPAL LCALSLALCA LSLPVRAATA SRGASQAGAP QGRVPEARPN |
| 51 | SMVVEHPEFL KAGKEPGLQI WRVEKFDLVP VPTNLYGDFF TGDAYVILKT |
| 101 | VQLRNGNLQY DLHYWLGNEC SQDESGAAAI FTVQLDDYLN GRAVQHREVQ |
| 151 | GFESATELGY FKSGLKYKKG GVASGFKHVV PNEVVVQRLF QVKGRRVVRA |
| 201 | TEVPVSWESF NNGDCFILDL GNNIHQWCGS NSNRYERLKA TQVSKGIRDN |
| 251 | ERSGRARVHV SEEGTEPEAM LQVLGPKPAL PAGTEDTAKE DAANRKLAKL |
| 301 | YKVSNGAGTM SVSLVADENP FAQGALKSED CFILDHGKDG KIFVWKGKQA |
| 351 | NTEERKAALK TASDFITKMD YPKQTQVSVL PEGGETPLFK QFFKNWRDPD |
| 401 | QTDGLGLSYL SSHIANVERV PFDAATLHTS TAMAAQHGMD DDGTGQKQIW |
| 451 | RIEGSNKVPV DPATYGQFYG GDSYIILYNY RHGGRQGQII YNWQGAQSTQ |
| 501 | DEVAASAILT AQLDEELGGT PVQSRVVQGK EPAHLMSLFG GKPMIIYKGG |
| 551 | TSREGGQTAP ASTRLFQVRA NSAGATRAVE VERKAGALNS NDAFVLKTPS |
| 601 | AAYLWVGTGA SEAEKTGAQE LLRVLRAQPV QVAEGSEPDG FWEALGGKAA |
| 651 | YRTSPRLKDK KMDAHPPRLF ACSNKIGRFV IEEVPGELMQ EDLATDDVML |
| 701 | LDTWDQVFVW VGKDSQEEEK TEALTSAKRY IETDPANRDR RTPITVVKQG |
| 751 | FEPPSFVGWF LGWDDDYWSV DPLDRAMAEL AA |
| Spot 98 SEQ ID. 2 | |
| 1 | MAPHRPAPAL LCALSLALCA LSLPVRAATA SRGASQAGAP QGRVPEARPN |
| 51 | SMVVEHPEFL KAGKEPGLQI WRVEKFDLVP VPTNLYGDFF TGDAYVILKT |
| 101 | VQLRNGNLQY DLHYWLGNEC SQDESGAAAI FTVQLDDYLN GRAVQHREVQ |
| 151 | GFESATFLGY FKSGLKYKKG GVASGFKHVV PNEVVVQRLF QVKGRRVVRA |
| 201 | TEVPVSWESF NNGDCFILDL GNNIHQWCGS NSNRYERLKA TQVSKGIRDN |
| 251 | ERSGRARVHV SEEGTEPEAM LQVLGPKPAL PAGTEDTAKE DAANRKLAKL |
| 301 | YKVSNGAGTM SVSLVADENP FAQGALKSED CFILDHGKDG KIFVWKGKQA |
| 351 | NTEERKAALK TASDFITKMD YPKQTQVSVL PEGGETPLFK QFFKNWRDPD |
| 401 | QTDGLGLSYL SSHIANVERV PFDAATLHTS TAMAAQHGMD DDGTGQKQIW |
| 451 | RIEGSNKVPV DPATYGQFYG GDSYIILYNY RHGGRQGQII YNWQGAQSTQ |
| 501 | DEVAASAILT AQLDEELGGT PVQSRVVQGK EPAHLMSLFG GKPMIIYKGG |
| 551 | TSREGGQTAP ASTRLFQVRA NSAGATRAVE VLPKAGALNS NDAFVLKTPS |
| 601 | AAYLWVGTGA SEAEKTGAQE LLRVLRAQPV QVAEGSEPDG FWEALGGKAA |
| 651 | YRTSPRLKDK KMDAHPPRLF ACSNKIGRFV IEEVPGELMQ EDLATDDVML |
| 701 | LDTWDQVFVW VGKDSQEEEK TEALTSAKRY IETDPANRDR RTPITVVKQG |
| 751 | FEPPSFVGWF LGWDDDYWSV DPLDRAMAEL AA |
| Spot 288 SEQ. ID 3 | |
| 1 | MKRVLVLLLA VAFGHALERG RDYEKNKVCK EFSHLGKEDF TSLSLVLYSR |
| 51 | KFPSGTFEQV SQLVKEVVSL TEACCAEGAD PDCYDTRTSA LSAKSCESNS |
| 101 | PFPVHPGTAE CCTKEGLERK LCMAALKHQP QEFPTYVEPT NDEICEAFRK |
| 151 | DPKEYANQFM WEYSTNYEQA PLSLLVSYTK SYLSMVGSCC TSASPTVCFL |
| 201 | KERLQLKHLS LLTTLSNRVC SQYAAYGEKK SRLSNLIKLA QKVPTADLED |
| 251 | VLPLAEDITN ILSKCCESAS EDCMAKELPE HTVKLCDNLS TKNSKFEDCC |
| 301 | QEKTAMDVFV CTYFMPAAQL PELPDVRLPT NKDVCDPGNT KVMDKYTFEL |
| 351 | SRRTHLPEVF LSKVLEPTLK SLGECCDVED STTCFNAKGP LLKKELSSFI |
| 401 | DKGQELCADY SENTFTEYKK KLAERLKAKL PEATPTELAK LVNKRSDFAS |
| 451 | NCCSINSPPL YCDSEIDAEL KNIL |
| Spot 289 SEQ. ID 4 | |
| 1 | MKRVLVLLLA VAFGHALERG RDYEKNKVCK EFSHLGKEDF TSLSLVLYSR |
| 51 | KEPSGTFEQV SQLVKEVVSL TEACCAEGAD PDCYDTRTSA LSAKSCESNS |
| 101 | PFPVHPGTAE CCTKEGLERK LCMAALKHQP QEEPTYVEPT NDEICEAFRK |
| 151 | DPKEYANQFM WEYSTNYEQA PLSLLVSYTK SYLSMVGSCC TSASPTVCFL |
| 201 | KERLQLKHLS LLTTLSNRVC SQYAAYGEKK SRLSNLIKLA QKVPTADLED |
| 251 | VLPLAEDITN ILSKCCESAS EDCMAKELPE HTVKLCDNLS TKNSKFEDCC |
| 301 | QEKTAMDVFV CTYFMPAAQL PELPDVRLPT NKDVCDPGNT KVMDKYTFEL |
| 351 | SRRTHLPEVF LSKVLEPTLK SLGECCDVED STTCFNAKGP LLKKELSSFI |
| 401 | DKGQELCADY SENTFTEYKK KLAERLKAKL PEATPTELAK LVNKRSDFAS |
| 451 | NCCSINSPPL YCDSEIDAEL KNIL |

### Example 6

### Calculation of the Vol % Ratio of the Spots ID 288 and 289

The combination of the Vol % values of the spots ID 289 and 288 can be expressed by two linear functions:

f1=33.813*(Vol % spot ID 288)+48.942*(Vol % spot ID 289)−7.155;

f2=103.508*(Vol % spot ID 288)+42.871*(Vol % spot ID 289)−15.248;

and represents a tool to correctly cluster (100%) the entire population into two main groups (groups G and Z) and to identify the subject MS 48 as an outlier (FIG. 7). The straight line separating the two clusters is defined by the function y=1.5629x−1.7155.

The identification of the spots ID 288 and 289 by mass spectrometry and the difference of the % volume values were validated by a 2-DE western blotting technique on a pool of samples.

In order to perform the 2DE Western blotting experiments, the cerebral fluid proteins were denatured, then separated on 2DE gel, as described above.

After the run, the gels were immediately immersed in an aqueous solution consisting in Tris 25 mM, 40 mM 6-aminohexanoic acid, and 20% v/v methanol, ensuring a final pH value of 9.4. The thus-separated proteins were transferred on nitrocellulose membranes (Hybond C-extra, with pores of 0.45 micrometers; GE Healthcare, Uppsala, Sweden) applying a “semi-dry” type transfer. After transferring, the membranes were incubated for at least 15 hours at 4° C. in a blocking solution composed of TBS (in the presence of 0.1% w/v Tween 20 (T-TBS) and 5% w/v of bovine serum albumin. TBS-T was further used in the washing steps, to remove possible non-specific bonds with the antibody. As the main recognition antibody, a goat polyclonal anti-DBP antibody (SIGMA ALDRICH) was used at a 1:500 dilution in 0.1% TBST.

As the secondary detection antibody, a HRP-conjugated (horseradish peroxidase) anti-goat was used at a 1:10.000 dilution in 0.1% TBST; the membrane was then incubated with a specific chemoluminescent substrate provided by the ECL Western Blotting kit (Pierce, Euroclone). Images relating to the proteins displayed following exposure on film were taken through the ImageMaster Labscan V3.0 (GE Healthcare, Uppsala, Sweden).

Subsequently, phosphoproteomic assays were conducted, to assess the possible involvement of the phosphorylation state in the different expression of the spots among the patients under examination. The gels were stained with ProQ Diamond (Molecular Probes Inc, Eugene, Oreg.) [Agrawal and Thelen, 2005) before being stained with Sypro Ruby, but no phosphorylation was shown in the identified spots.

By the 2-DE technique, allowing a separation of the proteins based on their isoelectric point, it has been shown that the differential migration of the spots ID 288 and 289 in the horizontal direction was due to their different glycosylation degree. In particular, the spot migrated in a more acidic position (ID 289) would contain the glycosylated isoform.

### BIBLIOGRAPHY

Agrawal G. K., Thelen J. J. “Development of a simplified, economical polyacrylamide gel staining protocol for phosphoproteins” Proteomics 5:4684-4688 (2005).

Bowser R., Cudkowicz M., Kaddurah-Daouk R. “Biomarkers for amyotrophic lateral sclerosis” Expert Rev. Mol. Diagn. 6: 387 (2006).

Disanto G., Ramagopalan S. V., Para A. AND., Handunnetthi L. “The emerging role of vitamin D binding protein in multiple sclerosis” J. Neurol. 258(3): 353-8 (2011).

Freedman M. S. “Long-term follow-up of clinical trials of multiple sclerosis therapies” Neurology 76 (1 Suppl 1): S26-34 (2011).

Ghezzi A. Cazzullo C. L. et al. “La Sclerosi Multipla ad onset precoce” Atti Congresso SINPI, S. Marino (1980).

Kulakowska A., Drozdowski W., Sadzynski A., Bucki R., Janmey P. A. “Gelsolin concentration in cerebrospinal fluid from patients with multiple sclerosis and other neurological disorders” European J. Neurology 15: 584-588 (2008).

Kulakowska A., Ciccarelli N. J., Wen Q., Mroczko B., Drozdowski Szmitkowski M., Janmey P. A., Bucki R. “Hypogelsolinemia, a disorder of the extracellular actin scavenger system, in patients with multiple sclerosis” BMC Neurology 10: 107 (2010).

Lacomblez L., Bensimon G., Leigh P. N., Guillet P., Meininger V. “Dose-ranging study of riluzole in amyotrophic lateral sclerosis” Amyotrophic Lateral Sclerosis/Riluzole Study Group II. Lancet, 347: 1425 (1996).

Lublin F. D., Reingold S. C. “Defining the clinical course of multiple sclerosis: results of an international survey”. National Multiple Sclerosis Society (USA) Advisory Committee on Clinical Trials of New Agents in Multiple Sclerosis. Neurology 46(4): 907-11 (1996).

Poser C., et. al. “The course of multiple sclerosis” Archives of Neurology 42: 10-35 (1985).

McDonald W I, Compston A, Edan G, Goodkin D, Hartung H P, Lublin F D, McFarland H F, Paty D W, Polman C H, Reingold S C, Sandberg-Wollheim M, Sibley W, Thompson A, van den Noort S, Weinshenker B Y, Wolinsky J S. “Recommended diagnostic criteria for multiple sclerosis: guidelines from the International Panel on the diagnosis of multiple sclerosis” Ann. Neurol. 50: 121-127 (2001).

Rithidech K. N., Honikel L., Milazzo M., Madigan D., Troxell R., Krupp L. B. “Protein expression profiles in pediatric multiple sclerosis: potential biomarkers” Mult. Scler. 15(4):455-64 (2009).

Robotti A., Natale M., Giuliano Albo A., Lis K., Perga S., Marnetto F., Gilli F., Bertolotto A. “Acute-phase proteins investigation based on lectins affinity capture prior to 2-DE separation: Application to serum from multiple sclerosis patients” Electrophoresis 17: 2882-2893 (2010).

Shaw P. J. and Williams R. “Serum and cerebrospinal fluid biochemical markers of ALS”. Amyotroph Lateral Scler Other Motor Neuron Disord. 1: S61 (2000).

1. A method of diagnosis and assessment of the progression status of Multiple Sclerosis, comprising:
i. measuring the levels of Vitamin D Binding Protein ID 289, glycosylated isoform (SEQ. ID 4) and Vitamin D Binding Protein ID 288 (SEQ. ID 3) in a sample C;
ii. obtaining the coordinates f1 and f2, where f1=33.813*ID 288 level+48.942*ID 289 level−7.155 and f2=103.508*ID 288 level+42.871*ID 289 level−15.248;
iii. positioning in the Cartesian plane the point C(f1, f2) and assessing the position thereof relative to the straight line y=1.5629x−1;
iv. where C(f1, f2)>y=1.5629x−1, the sample C will be assigned to the MSSP (secondary progressive MS) or MSPP (primary progressive MS) group; where C(f1, f2)<y−1.5629x−1, the sample C will be assigned to the MSRR group (relapsing-remitting MS).

2. A method of assessment of the progression status of Multiple Sclerosis, comprising:
i. measuring the ID 97 (SEQ. ID 1) and/or ID 98 (SEQ. ID 2) level in a sample C and in a control MSRR;
ii. obtaining the value G=ID 97 level+ID 98 level, or G=ID 97 level or G=ID 98 level in the sample G_{c} and in the control G_{ctrlMSRR};
iii. where G_{c}>G_{ctrlMSRR} in a statistically significant manner, assigning the sample C to the MSSP (secondary progressive MS) or MSPP (primary progressive MS) group; where G_{c}=G_{ctrlMSRR}, assigning the sample C to the MSRR group (relapsing-remitting MS).

3. The method in accordance with the claim 1, further comprising:
i. measuring the ID 97 (SEQ. ID 1) and/or ID 98 (SEQ. ID 2) level in a sample C and in a control MSRR;
ii. obtaining the value G=ID 97 level+ID 98 level, or G=ID 97 level or G=ID 98 level in the sample (G_{c}) and in the control (G_{ctrlMSRR});
iii. where C(f1, f2)>y=1.5629x−1 and G_{c}>G_{ctrlMSRR} in a statistically significant manner, assigning the sample C to the MSSP MSPP group; where C(f1, f2)<y=1.5629x−1 and G_{c}=G_{ctrlMSRR}, assigning the sample C to the MSRR group.

4. The method in accordance with claim 1, further comprising the assessment of further biomarkers selected in the group comprising: complement factor C3, complement factor C4, beta-2-microglobulin, Clusterin, Prostaglandin H2 D isomerase, Haptoglobin, Immunoglobulins, Apolipoprotein E, Beta fibrinogen, alpha 2 macroglobulin, Fetuin A, serin protease inhibitor, Tansthyretin, Albumin, Transferrin, Apolipoprotein D, Retinol binding protein, Apolipoprotein A4, SPARC-like protein, Autotaxin t, Pigment epithelium derived factor, Angiotensinogen, Chromogranin A, Tuberous sclerosis complex 2, Cystatin C, Ceruloplasmin, Superoxide dismutase 1, Actin, Beta V Spectrin, Cartilage acidic protein 1, Fibulin 1, Calsyntenin 3, Contactin 1, Xantin dehydrogenase/oxidase, RNA binding motif protein 7, Ribonuclease 1, neuroendocrine protein 7 B2, alpha 1 Antitrypsin, alpha 1 Antichymotrypsin, Kallikrein 6, EGF-containing fibulin-like extracellular matrix protein 1, Plasminogen, Antithrombin III, Dickkofp relata protein 3, neuronal pentraxin receptor, Tetranectin.

5. The method according to claim 1, where said proteins are detected and quantified by techniques for protein analysis, preferably selected from the group comprising Western-Blot, 2DE gel electrophoresis, ELISA (Enzyme-Linked Immunosorbent assay), RIA (Radioimmunoassay), Competitive EIA (Competitive Enzyme Immunoassay), DAS-ELISA (Double Antibody Sandwich-ELISA), other immunocytochemical or immunohistochemical techniques optionally combined with HPLC or mass spectrometry.

6. A kit for implementing the method in accordance to claim 1, comprising two antibodies capable of specifically recognizing the two isoforms of Vitamin D Binding protein SEQ. ID 3 and SEQ. ID 4, and/or one or two antibodies capable of recognizing in a combined or selective manner the gelsolin isoforms SEQ. ID 1 and SEQ. ID 2, and optionally one or more antibodies capable of recognizing one or more of the biomarkers selected from the group comprising complement factor C3, complement factor C4, beta-2-microglobulin, Clusterin, Prostaglandin H2 D isomerase, Haptoglobin, Immunoglobulins, Apolipoprotein E, Beta fibrinogen, alpha 2 macroglobulin, Fetuin A, serin protease inhibitor, Tansthyretin, Albumin, Transferrin, Apolipoprotein D, Retinol binding protein, Apolipoprotein A4, SPARC-like protein, Autotaxin t, Pigment epithelium derived factor, Angiotensinogen, Chromogranin A, Tuberous sclerosis complex 2, Cystatin C, Ceruloplasmin, Superoxide dismutase 1, Actin, Beta V Spectrin, Cartilage acidic protein 1, Fibulin 1, Calsyntenin 3, Contactin 1, Xantin dehydrogenase/oxidase, RNA binding motif protein 7, Ribonuclease 1, neuroendocrine protein 7 B2, alpha 1 Antitrypsin, alpha 1 Antichymotrypsin, Kallikrein 6, EGF-containing fibulin-like extracellular matrix protein 1, Plasminogen, Antithrombin III, Dickkofp relata protein 3, neuronal pentraxin receptor, Tetranectin, and optionally reagents for the detection of said antibodies bound to said proteins.

7. A compound capable of inducing the isoform of the Vitamin D binding protein SEQ. ID 4 for use in the treatment of MS, where said compound is selected from the group comprising: a recombinant vector selected from the vectors that are integrated or not integrated in the genome, characterized in that said recombinant vector comprises a gene coding for the sialidase or a homologous thereof, or contains an element capable of acting at the level of the endogenous promoter of said sialidase by activating it; isopropyl-b-D-thiogalactopyranoside (IPGT); lactose; interferon.

8. A composition comprising one or more of the compounds according to the claim 7 and excipients pharmacologically acceptable for use in MS.


SEQUENCE LISTING (4 sequences)

SEQ ID NO: 1 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 2 - protein, 782 aa, Homo sapiens; identical to SEQ ID NO: 1

SEQ ID NO: 3 - protein, 474 aa, Homo sapiens [sequence omitted]

SEQ ID NO: 4 - protein, 474 aa, Homo sapiens [sequence omitted]